(19) United States
(12) Patent Application Publication
(10) Pub. No.: US 2013/0012964 A1
(43) Pub. Date: Jan. 10, 2013

(54) PORT CLOSURE DEVICE AND METHOD

(76) Inventor: Steven Warnock, Salt Lake City, UT (US)

(21) Appl. No.: 13/541,533

(22) Filed: Jul. 3, 2012

Related U.S. Application Data
(60) Provisional application No. 61/505,253, filed on Jul. 7, 2011.

(51) Int. Cl. A61B 17/04

(52) U.S. Cl. 606/145

(57) ABSTRACT

A surgical device for creating a secure closure of a laparoscopic port opening or similar wound. The device may employ a tissue grasping notch that grasps the fascia layer as a suture-bearing needle is deployed from the distal end of the device through the fascia layer and into a needle-capture mechanism. The fascia layer may then be released, the device turned and the fascia layer grasped and a second suture-bearing needle deployed. The two needles may be connected by a suture, and as the device is removed from the wound, the suture extends through the fascia on one side, across the wound on the deep side, and through the fascia on the other side, thus creating a secure closure of the port wound through the fascia layer.

### PRIORITY CLAIM

The present application claims priority to U.S. Provisional Patent Application Ser. No. 61/505,253, filed Jul. 7, 2011, which is expressly incorporated herein in its entirety.

### BACKGROUND OF THE INVENTION

1. The Field of the Invention

The present invention relates to a surgical suturing device and method. More specifically, the present invention relates to a device for closing the puncture wound of a laparoscopic access port or similar puncture wound.

2. State of the Art

Advancements in the field of surgery have led to minimally invasive surgical techniques that have great benefits for patients. Such techniques use very small incisions created by a surgical trocar, through which surgical instruments and visualization tools are inserted. Often laparoscopes are used for visualization. Laparoscopic surgery has multiple advantages over traditional surgery, including less pain and disfigurement, and reduced patient recovery time, which consequently permits the patient to return to normal activity in a shorter period of time.

However, there is a substantial clinical disadvantage with laparoscopic surgery and other minimally invasive techniques. While the puncture wound is very small and has the advantages discussed above, the small opening gives the surgeon very little room to engage tissues below the epidermis, thus often making the puncture wound difficult to close. This task is made even more difficult when the patient is obese, and the puncture wound is relatively deep relative to its cross-sectional area. An obese patient often requires the puncture site to be enlarged to adequately close it, negating some of the advantages of laparoscopic surgery.

Additionally, if the closure is not made complete, significant medical complications may ensue. For example, if the closure of the trocar incision is only skin-deep, and does not include the deep layers of the fascia below the skin, there is a risk of delayed bleeding or herniation. According to the present state of the art, closure of the port wound relies heavily on the skill level of the surgeon, and skill and experience of surgeons varies substantially.

Accordingly, there is a substantial need in the art for a laparoscopic fascial closure tool or suturing device, and a method of use. Specifically, a device is needed that is able to ensure the suture goes through the deep fascia layer, and is capable of closing port wounds of various sizes. The device may also be simple to deploy in surgery so it may be used by surgeons of different skill levels.

### SUMMARY OF THE INVENTION

It is an object of the present invention to provide a device for facilitating the closure of surgical trocar wounds or puncture wounds. It is another object of the present invention to provide a method for closing such wounds. In accordance with the principles of the present invention, a device and method are disclosed which facilitate closure of a puncture or trocar wound.

In one aspect of the invention, a device is provided that assists in the closing of interior tissue layers of a trocar wound. Because it is important to fully close a trocar wound including the fascia layer, and not just the skin, the invention may have an aspect that provides for a tissue grasping notch and lever. The notch and lever ensure the fascia layer is grasped and held, and the subsequent suture passes through the deep fascia layer.

In accordance with another aspect of the invention, the device and method are configured to provide a suture that can cover the periphery of a variety of different-sized trocar or puncture wounds.

According to another aspect of the invention, the device may be simple to use and may be used in surgery by surgeons of nearly any skill level.

According to the present method, the device may be deployed during surgery by inserting the distal end of the device into the wound to be sutured. The device is then moved into and out of the wound until the fascia layer is caught in a tissue grasping notch disposed along the device. The fascia is then secured by closing a tissue grasping lever. The surgeon then pulls the activation trigger, deploying a suture-bearing needle from the distal end of a port arm, through the fascia, and into a proximal portion of the port arm where a capture mechanism collects and secures the needle.

The tissue grasping lever then may be released, opening the tissue grasping notch. The device may then be turned a desired angle (e.g. 90, 120 or 180 degrees depending on the number of sutures used) within the wound and the process may be repeated, grasping the fascia layer, closing the tissue grasping lever, deploying a suture-bearing needle through the fascia, and then releasing the tissue grasping lever. When a desired number of sutures have been placed, the device may then be removed from the wound, leaving behind one or more sutures that more effectively close the laparoscopic port opening.

These and other aspects of the present invention are realized in the device and method as shown and described in the following figures and related description.

### BRIEF DESCRIPTION OF THE DRAWINGS

Various embodiments of the present invention are shown and described in reference to the numbered drawings wherein:

FIG. 1 shows a side-view of the closure device constructed in accordance with one aspect of the present invention, with the slider in an open position;

FIG. 2 shows a magnified, plan view of the distal end of the port arm of the device depicted in FIG. 1;

FIG. 3 shows a side, plan view of the device depicted in FIG. 1;

FIG. 3A shows a close-up view of a needle and capture mechanism which uses a curved needle;

FIGS. 4A-4C show magnified views of the needle-driving mechanism, needle, and needle-capturing mechanism of the device depicted in FIG. 1;

FIG. 5 shows a representation of a cross-sectional view depicting placement of the looped suture following withdrawal of the laparoscopic port closure device from a patient's body; and

FIG. 6 shows a side, partial, cut-away view of an alternate configuration for the device according to the present invention.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single FIGURE, and as such, multiple figures are presented to separately illustrate the various details of the invention in greater clarity. Similarly, not every embodiment need accomplish all advantages or aspects of the present invention.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the accompanying claims. It is understood that the present invention includes any alterations and modifications to the illustrated embodiments and includes further applications of the principles of the invention as would normally occur to one skilled in the art to which the invention pertains. Also, although the present invention is described primarily in reference to laparoscopic surgery, one of skill in the art would appreciate that it may be applied to other surgical procedures and may be used in the surgical closure of puncture wounds or other deep wounds.

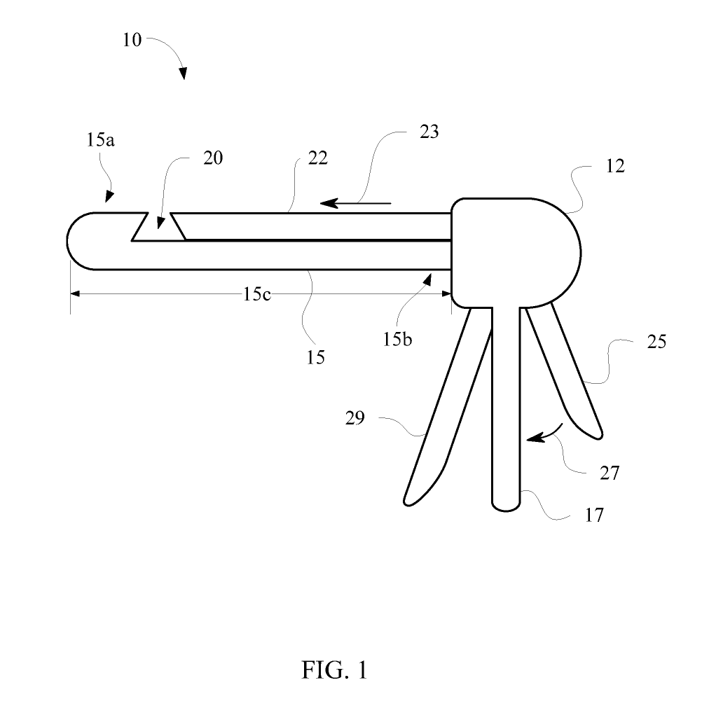

FIG. 1 shows a port closure device, generally indicated at 10, made in accordance with the present invention. The device may be formed of a body 12, consisting generally of a port arm or cannula member 15 and a handle member 17. The port arm 15 has a distal end 15a and a proximal end 15b, with a length 15c. The distal end 15a may be configured for insertion into a patient, and the length 15c may be such that when the distal end 15a is inserted in the innermost tissue layer of a patient, the proximal end 15b is accessible outside the patient. As an example, the length 15c may be approximately 15 centimeters, but one of skill in the art would appreciate that the device could be configured with numerous other lengths.

On an exterior surface of the port arm 15, a notch 20 may be provided. The notch may be, for example, about 4 centimeters from the distal end 15a of the port arm. It is appreciated that the notch 20 may also be disposed at any other desired position along the port arm. The notch 20 may be configured to function as a tissue-grasping notch and can be “opened” or “closed” by the action of a slider 22 operated by a tissue-grasping lever 25. In other words the length of the notch can be changed by use of the grasping lever to selectively grasp tissue. The movement of the slider 22 distally, as indicated by arrow 23, “closes” the notch 20, i.e. lessens the length so that the arm and the slider grasp opposing sides of the tissue(s) in the notch.

A tissue-grasping lever 25 may be located on a portion of the handle 17. The tissue-grasping lever 25 may have an open position and a closed position, corresponding to the opening and closing of the tissue-grasping notch 20. FIG. 1 depicts the device with the tissue grasping notch 20 in an open position. As the tissue-grasping lever is moved from an opened position towards a closed position, as indicated by the arrow 27 in FIG. 1, the slider 22 is moved toward the distal end 15a of the port arm 15, as indicated by the arrow 23 in FIG. 1, thus closing the tissue-grasping notch 20. Thus, as used herein, reference to the notch having an open position indicates that the slider is moved to enlarge the size of the notch and reference to a closed position indicates that the slider is moved to reduce the size of the notch.

Likewise, as the tissue-grasping lever 25 may be referred to as being moved from a closed position to an opened position based on its effects on the slider. Thus, the lever 25 can be moved from the open position to the closed position so that the slider 22 slides out of or away from the body 12 of the device towards the distal end 15a of the port arm 15. In FIG. 1, this motion would include the slider 22 moving in the direction indicated by the arrow. Likewise, the lever 25 can be moved from the closed position to the open position so that the slider retracts into or toward the body 12 of the device. One of skill in the art will appreciate that the tissue-grasping notch 20, slider 22, and tissue-grasping lever 25 may be provided at different positions on the device and still achieve similar results.

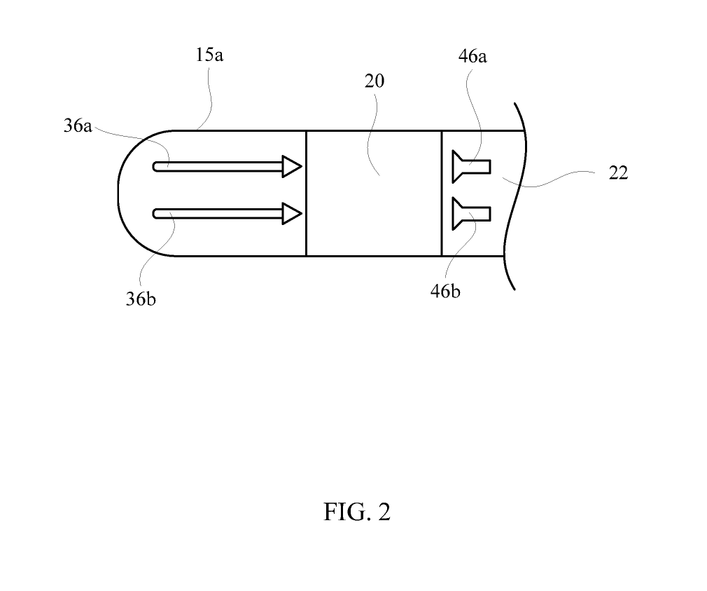

Turning now to FIG. 2, a magnified view of the distal end 15a of the port arm is shown, with the top of the port arm cut-away to view the inside of the arm. The slider 22 may also be provided with one or more needle capture mechanisms, 46a and 46b. Typically, at least two needle capture mechanisms will be used. However, one needle capture mechanism or multiple needle capture mechanisms may be used. As the tissue-grasping notch 20 is closed by the movement of the slider 22 distally towards the distal end 15a of the port arm, the needle capture mechanisms 46a, 46b, located within the needle capture mechanism 22, also move distally.

One or more needles 36a, 36b (with two or more being most common), are provided in the distal end of the port arm, with the needles pointing generally towards the proximal end 15b of the device. As the tissue-grasping notch 20 is closed, the needle capture mechanisms 46a, 46b move into close proximity with the needles 36a, 36b. These needle-capture mechanisms 46a, 46b accept the suture-bearing needles 36a, 36b, respectively and secure the needles after they have been deployed as will be further described below. Thus, the needle capture mechanisms 46a, 46b receive and hold the needles 36a, 36b as the device 10 is withdrawn from the wound to thereby place a suture. It will be appreciated that the needles 36a and 36b could be placed simultaneously or sequentially depending on the design of the device. For example, the capture mechanism 46a could be placed ahead of the capture mechanism 46b so that during the first use the needle 36a is deployed and during a second use the needle 36b is deployed. Likewise, the actuator mechanism could be configured to only advance one needle at a time.

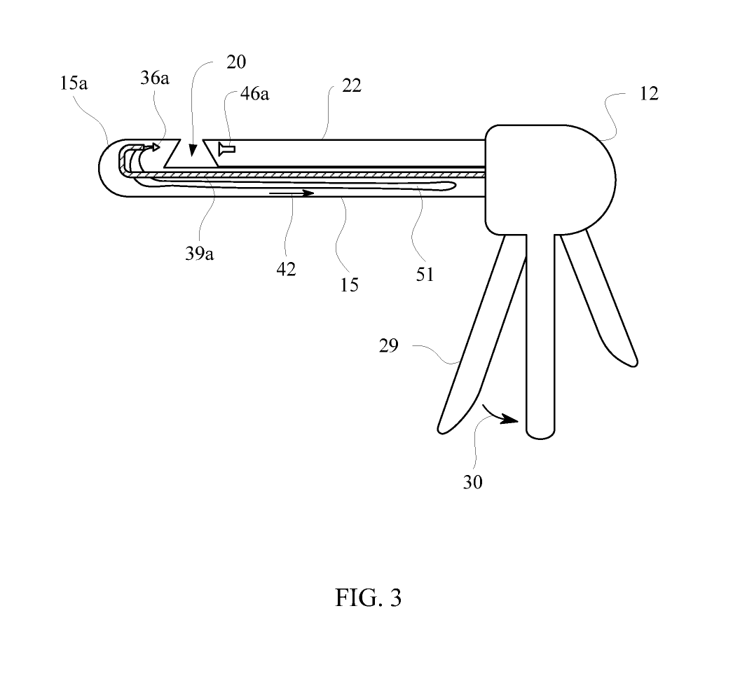

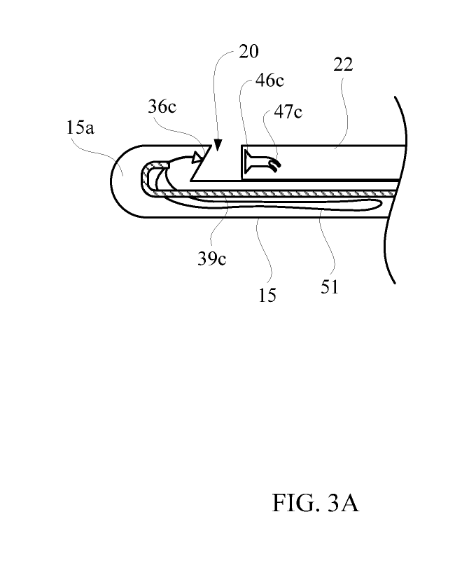

Turning now to FIG. 3, a side, plan view of the device is shown. The needle 36a within the distal end of the port arm 15b may be attached to a needle driving mechanism 39a, which extends generally from a position adjacent the distal end of the port arm 15a, through the length of the arm, to a position adjacent an activation trigger 29. As the activation trigger 29 is pulled, as indicated by arrow 30 in FIG. 3, the needle driving mechanism 39a slides proximally towards the body 12 of the device, as indicated by arrow 42. This action drives the needle 36a through the space of the tissue-grasping notch 20 and any tissue disposed therein, and into the needle-capturing mechanism 46a. This action will be described in further detail below.

The activation trigger 29 may be positioned on the device similar to the position of a trigger on a gun, on the distal side of the handle 17, and may be positioned to be emanating from the junction of the handle 17 and port arm 15. It can also be placed on other parts of the device. Likewise, the “activation trigger” can be a slide, crank, knob, lever or other structure for moving the drive mechanism and need not resemble the trigger of a gun.

The activation trigger 29 may be designed larger than the typical trigger to facilitate greater control and leverage. In accordance with one aspect of the invention, the activation trigger 29 or cooperating structures may be configured so that the activation trigger will not deploy unless the tissue-grasping notch 20 is closed (i.e. the slide 22 has moved distally), and the tissue-grasping lever 25 is in the corresponding closed position. As explained below, this ensures that the device does not deploy until it is in the proper position within the subcutaneous tissue of the patient.

Within the port arm 15 may be one or more conduits. The conduits may extend substantially the entire length of the port arm 15. In one embodiment of the invention, three conduits are used. Two outer conduits may be substantially identical and may be configured to house the needle-driving mechanisms 39a, 39b which hold, deploy, and release suture-bearing needles 36a, 36b, respectively. The center conduit may be configured to house looped suture 51 that is attached to each of the two needles 36a, 36b. One of skill in the art will appreciate that the three conduits could be configured in a different arrangement and still achieve the goals of the present invention. For example, the three conduits may be aligned in a row, or two conduits may be stacked on top of a third.

The needles 36a, 36b may be held in a position just distal to the tissue grasping notch 20 and within the distal end of the arm 15b. Consequently, the most distal portion of the port arm 15b and the needles 36a, 36b contained therein will remain still and protected by the device as the tissue grasping notch 20 opens and closes due to the action of the more proximal slider 22. When the activation trigger 29 is pulled, one or more of the needles is pushed proximally through the tissue held in the tissue grasping notch 20.

While a straight needle is generally described, in one embodiment of the invention, a curved needle and a needle delivery mechanism may be used as shown in FIG. 3A. The curved needle 36c may allow the device to get a more substantial bite of tissue in the grasping notch 20 as the needle is passed from the port arm 15 into the needle capturing mechanism 46c in the slide 22 (or vice versa, as the needle could be passed from the slide 22 into a needle capturing mechanism located in the port arm). One having skill in the art would appreciate that numerous types of needles and their corresponding capturing mechanisms may be used in accordance with the present invention.

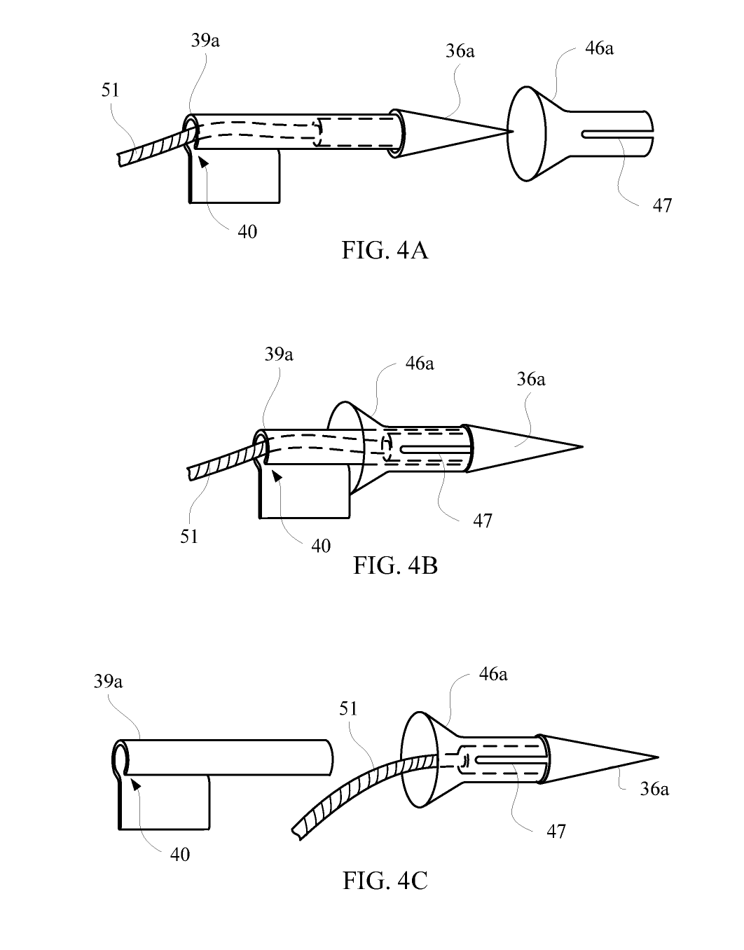

FIGS. 4A, 4B, and 4C show magnified views of how a needle 36a is driven through the tissue grasping notch and into the needle-capturing mechanism 46a. FIG. 4A shows the needle attached to the needle-driving mechanism in a position in which the needle would be advancing through the tissue, but before it has been inserted into the needle capture mechanism. FIG. 4B shows the needle and needle-driving mechanism as they are inserted into the needle capture mechanism. FIG. 4C shows the needle within the needle capture mechanism, with the needle-driving mechanism retracted. In use, the needle 36a will also be driven through the fascia layer disposed within the tissue-grasping notch, but the fascia layer and grasping notch are omitted from the figures for purposes of illustration.

In FIG. 4A, the needle is shown as it is held by the needle-driving mechanism. The looped suture 51 may be attached to or adjacent to the distal end of the needle 36a. The tip of the needle may protrude from the needle-driving mechanism 39a. The needle-driving mechanism 39a may be circular in shape or other appropriate configuration, with a gap or slot 40 near the bottom of the shape. This gap 40 allows the suture 51 to exit the needle-driving mechanism 39a as soon as the needle-driving mechanism is pulled away after the needle 36a is held by the needle-capturing mechanism.

The needle-capture mechanism 46a may have a flared opening to receive the needle. In accordance with one aspect of the invention, the needle-capture mechanism may be constructed of a metal or durable plastic, and may include a small, cut-away slot 47. This slot would allow the needle-capture mechanism to have some give as the slightly-wider needle tip is inserted, and then close again tightly to prevent the needle from coming out.

Turning now to FIG. 4B, the needle tip has been inserted, via the needle-driving mechanism 39a, into or through the needle-capturing mechanism 46a. The needle-driving mechanism 39a may have a diameter slightly smaller than the diameter of the needle-capturing mechanism 46a, such that it may be inserted into the needle-capturing mechanism. It may be preferred to provide the largest diameter of the needle 36a, often at the base of the needle tip, with a diameter larger than the diameter of the needle-capturing mechanism 46a. This allows the needle-driving mechanism 39a to insert the needle 36a into the needle-capturing mechanism 46a, with the tip of the needle being inserted past the end of the needle-capturing mechanism. The slightly larger diameter of the needle tip prevents the needle from retracting with the needle-driving mechanism 39a. The needle-driving mechanism can move in and out freely within the needle capture mechanism such that once the needle has been captured, the needle-driving mechanism can freely and easily retract.

Turning now to FIG. 4C, the needle-driving mechanism 39a is shown retracted from the needle-capturing mechanism 46a, leaving the needle tip securely in place with the edge of the needle tip in contact with the end of the needle-capturing mechanism. The suture 51 has also withdrawn from the needle-driving mechanism 39a, slipping out from the gap 40 near the bottom of the needle-driving mechanism.

In use, the surgeon would insert the distal end 15b of the port arm 15 into the patient. The port arm 15 is then moved into and out of the patient until the fascia layer of the abdominal wall is disposed or caught within the tissue grasping notch 20. The surgeon may tell that the fascia is engaged by feel, or if desired, by vision using the laparoscope. Once the fascia layer is disposed in the tissue grasping notch 20, the fascia layer is secured within the notch by closing the notch 20 by movement of the slide 22. This is accomplished by pressing the tissue grasping lever 25 distally, thus moving the slider 22 distally to close the tissue grasping notch 20. As the notch closes, it will tend to displace softer tissue, while the fascia, which is more firm and sheet-like, will remain secured within the tissue-grasping notch. The device may also be configured to be sized such that the device fills the space within the port site, thus displacing any other tissue that may be in the way.

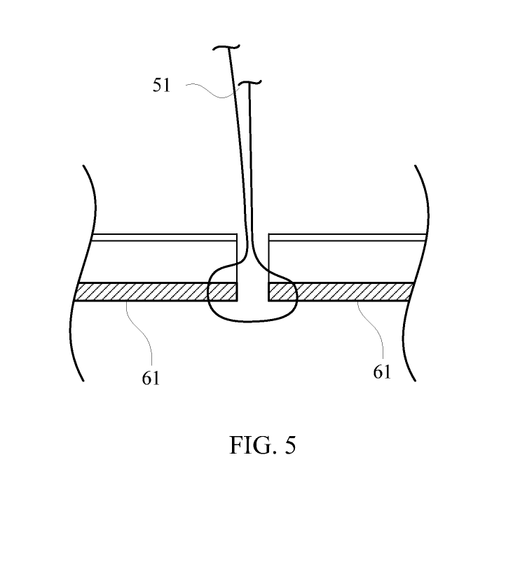

Upon securing the fascia layer within the tissue grasping notch 20 of the device, the surgeon then presses the activation trigger 29. This deploys a first needle 36a, driving the needle 36a from the distal end of the port arm 15b through the fascia layer in the tissue grasping notch 20, and into the capture mechanism 46a, which collects and secures the needle 36a and the accompanying suture. The activation trigger 29 is then released allowing the needle-driving mechanism 39a to retract into the distal end of the device 15b, leaving only the suture in place through the fascia layer. The surgeon then releases the fascia layer 61 (FIG. 5) by opening the tissue grasping notch 20. This is achieved by moving the tissue grasping lever 25 from the closed position to the open position, correspondingly sliding the mechanism 22 more proximally toward or into the body 12, thereby opening the tissue grasping notch 20.

Next, the device is turned a desired angle, typically 180 degrees within a port opening or wound when two needles are used, and the steps are repeated to deploy a second needle 36b. The surgeon moves the device in and out of the wound to catch the fascia layer 61 within the tissue grasping notch 20. The surgeon then secures the fascia layer by pressing the tissue grasping lever 25 into a closed position, thereby moving the slider 22 distally to close the tissue grasping notch 20. The activation trigger 29 is then pressed, deploying the second needle 36b. The needle 36b is driven from the distal end 15a of the port arm 15, through the fascia layer 61 within the tissue-grasping notch 20, and into the capture mechanism 46b, which collects and secures the needle 36b.

Again, the fascia layer is released by moving the tissue grasping lever 25 to an open position, sliding the mechanism 22 more proximally and opening the tissue grasping notch 20. With the fascia layer released and both needles deployed, the surgeon then withdraws the device 10 from the port opening. (While the above example and figures describe a procedure with two needles 36a, 36b, it will be appreciated that the device 10 could be made with addition needles if desired.)

The needles 36a, 36b are connected by a suture 51, or lines from each needle may be tied together. The suture may be, for example, a single-braided suture about 60 centimeters in length. As the device 10 is withdrawn from the patient's body, FIG. 5 depicts the placement of the looped suture. The suture 51 runs from the capture mechanism 46a within the device 10, into the wound, through the fascia 61 on one side of the fascial incision, across the incision on the deep surface of the fascia to the fascia on the other side of the incision, back through the fascia and out of the wound into the capture mechanism 46b. The suture 51 can then be cut and tied or secured by other mechanical means (for example, clips, zips, or clamps), or in any suitable manner the surgeon prefers, thus closing the fascial incision.

Additional embodiments of the current device could be employed. For example, the device could have the tissue-grasping notch and tissue-grasping lever located at different positions on the device. The activation trigger could also be placed at a different position to achieve the same end result. Similarly, the slider could be integrated with the port arm, or it could be separate. The tissue grasping notch may be configured of various sizes and shapes to grasp and hold tissue. The device may employ a single needle, with a single needle-driving mechanism and needle-capturing mechanism, or multiple needles, needle-driving mechanisms, and needle-capturing mechanisms. The needles may be straight, or if desired, curved needles with their corresponding needle capture mechanisms may be used.

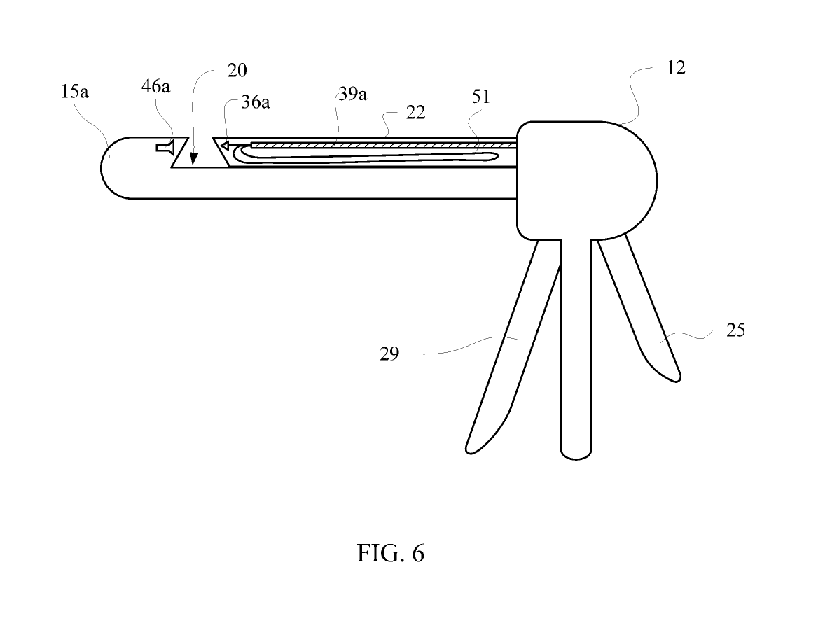

Additionally, the needle-driving mechanism could be located in the slide, with the needle-capture mechanism located in the distal end of the port arm, as seen in FIG. 6. Such a configuration would produce a suture that began on the deep side of the puncture wound, went across the shallow fascia, and again into the deep puncture would. Such a closure would be a “buried knot,” and the ends could be pulled tight and lifted out of the wound.

There is thus disclosed an improved device for closing laparoscopic port wounds. It will be appreciated that numerous changes may be made to the present invention without departing from the scope of the claims.

What is claimed is:

1. A surgical device comprising:
A body having an arm and a slide disposed for movement along the arm, the arm and slide cooperating so as to form a tissue securing notch of changeable length;
at least one needle disposed on one of the arm and the slide, the needle having a suture attached thereto;
and
at least one needle-capturing mechanism disposed on one of the arm and the slide for receiving and securing the needle.

2. The surgical device of claim 1, wherein the at least one needle comprises two needles and wherein the two needles are connected by a piece of suture material.

3. The surgical device of claim 2, wherein the two needles are located in a distal end of the arm, and wherein the at least one needle-capturing mechanism comprises two needle-capturing mechanisms located in a distal end of the slide.

4. The surgical device of claim 1 further comprising at least one needle driving mechanism for advancing the needle through the tissue securing notch.

5. The surgical device of claim 4, wherein the surgical device further comprises an activation trigger in communication with the needle driving mechanism for activating the needle driving mechanism to thereby advance the at least one needle.

6. The surgical device of claim 1, wherein the surgical device further comprises a grasping lever for moving the slide relative to the arm to thereby change the length of the tissue securing notch.

7. A surgical device for fashioning a closure of a laparoscopic puncture site or other puncture wound, comprising:
a body member;
an elongate cannula having a generally hollow lumen with proximal and distal portions, the proximal portion being connected to the body member;
at least two needle driving mechanisms disposed in the distal end of the elongate cannula, the at least two needle driving mechanisms each having a proximal and a distal end, and being configured to hold at least one needle on the distal end, and being connected to an activation trigger on the proximal end thereof operative to selectively cause the at least two needle driving mechanism to advance proximally or retract distally;
an elongate slider disposed along the cannula having a proximal and a distal end, the slider being connected to a actuator on a proximal portion of the canula and being operative to selectively cause the slider to advance distally or retract proximally within the body of device, and the slider containing at least two needle-capturing mechanisms in the distal portion thereof; and
a notch in the distal portion of the cannula member defined by the void between the distal portion of the cannula member and the distal end of the elongate slider.

8. The of surgical device of claim 7, further comprising at least two needles disposed in communication with the at least two needle driving mechanisms.

9. The surgical device of claim 8, wherein the at least two needles are connected by a piece of suture material.

10. The surgical device of claim 9, wherein the at least two needle driving mechanisms are operatively transitional between a first configuration wherein the needles are within the lumen of the cannula, and a second configuration wherein the needles are driven proximally into the at least two needle-capturing mechanisms.

11. The surgical device of claim 10, wherein said device is operative to deploy the at least two needles into the needle-capturing mechanisms of the elongate slider one-at-a-time, such that after both of the at least two needles are deployed, the device may be withdrawn from the body with the suture extending between the needles, forming a closure of the laparoscopic puncture site.

12. The surgical device according to claim 7, wherein the activation trigger connected to the at least two needle driving mechanisms is not operational until the elongate sliding member is advanced distally.

13. A method for closing a port wound comprising:
selecting a device having a port arm and a slider operating together to form a tissue retention notch;
disposing the device within the port wound;
moving the device within the wound to secure a first piece of tissue into the tissue retention notch;
moving the slider toward the port arm to secure the first piece of tissue;
advancing a first needle through the notch and securing the first needle; and
sliding the slider proximally to release the first piece of tissue.

14. The method according to claim 13, wherein the method further comprises the steps of:
turning the device;
moving the device within the wound to secure a second piece of tissue into the tissue retention notch;
sliding the slider toward the port arm to secure the second piece of tissue;
advancing a second needle through the notch and securing the second needle, the first and second needles having suture extending therebetween; and
extracting the device.

15. The method according to claim 13, wherein the selecting a device step further comprises selecting a device comprising
at least a first and second needle-driving mechanism, the first needle-driving mechanism holding the first needle, and the second needle-driving mechanism holding the second needle;
an activation trigger connected to the first and second needle-driving mechanisms, the activation trigger configured to move the first and second needle-driving mechanisms proximally; and
at least a first and second needle-capturing mechanism within the slider.

16. The method according to claim 15, wherein the step of advancing a first needle through the notch and securing the first needle comprises pressing the activation trigger a first time to drive the first needle through the fascia and into the first needle-capturing mechanism.

17. The method according to claim 15, wherein the step of advancing a second needle through the notch and securing the second needle comprises pressing the activation trigger a second time to drive the second needle through the fascia and into the second needle-capturing mechanism.

18. The method according to claim 13, wherein the step of moving the device within the wound to secure a first piece of tissue into the tissue retention notch comprises inserting the device into a patient and removing the device until the fascia layer of the patient is caught in the notch.

19. The method according to claim 18, wherein a surgeon discerns the first piece of tissue is secured in the tissue retention notch using feel.

20. The method according to claim 18, wherein the surgeon discerns the first piece of tissue is secured in the tissue retention notch using direct visualization through a laparoscope.

21. The method according to claim 17, wherein the step of extracting the device comprises withdrawing the device from the port wound, leaving the suture extending between the first needle and second needle, forming a closure of the port wound.

22. A body for closing a hole in tissue, the body comprising:
an elongate arm;
a slide disposed for movement along the arm, the arm and slide cooperating so as to form a tissue securing notch of changeable length;
at least one needle disposed on one of the arm and the slide, the needle having a suture attached thereto;
and at least one needle-capturing mechanism disposed on one of the arm and the slide for receiving and securing the needle.
